(19) United States
(12) Patent Application Publication
(10) Pub. No.: US 2026/0138971 A1
(43) Pub. Date: May 21, 2026

(54) PROCESS FOR SYNTHESIS OF RES METIROM AND ITS INTERMEDIATES THEREOF

(71) Applicant: ROYAL PHARMA, Palghar, Maharashtra (IN)

(72) Inventor: Nilesh Vipinchandra MEHTA, Mumbai, Maharashtra (IN)

(21) Appl. No.: 19/118,617

(22) PCT Filed: Feb. 23, 2023
(86) PCT No.: PCT/IN2023/050173

§ 371 Date: Apr. 4, 2025

(30) Foreign Application Priority Data
Oct. 21, 2022 (IN) 202221060345

(51) Int. Cl. C07D 403/12; C07D 237/14

(52) U.S. Cl. CPC C07D 403/12; C07D 237/14

(57) ABSTRACT

The present invention discloses a process for synthesis of Res metirom and its intermediates thereof. More particularly, the invention discloses process for synthesis of a key intermediate, 6-(4-amino, 2,6-dichlorophenoxy)4-isopropylpyridazin-3(2H)-one, of Res metirom via novel intermediate compounds of formula A. Wherein, R is selected from methyl, ethyl and propyl.

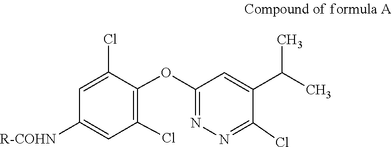

### TECHNICAL FILED

The invention relates to a process for synthesis of Res metirom and its intermediates thereof. More particularly, the invention relates to process for synthesis of a key intermediate, 6-(4-amino, 2,6-dichlorophenoxy)4-isopropylpyridazin-3(2H)-one, of Res metirom via a novel intermediate compounds of formula A.

### BACKGROUND AND PRIOR ART

Res metirom, a pyridazinone derivative useful as thyroid hormone receptor agonist, was first disclosed in WO2007009913, having the following structural formula.

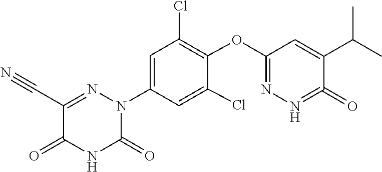

Res metirom (MGL-3196) is a liver-directed, orally active, selective thyroid hormone receptor-β agonist designed to improve NASH by increasing hepatic fat metabolism and reducing lipotoxicity.

WO2007009913 discloses synthesis of 6-(4-amino-2,6-dichlorophenoxy)-4-isopropylpyridazin-3(2H)-one, useful as an intermediate, for making pyridazinone compounds as thyroid hormone analogs, by the process shown in scheme 1 below.

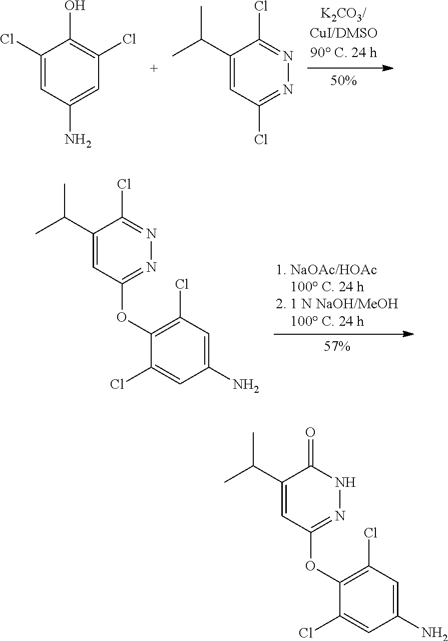

According to the above scheme, 4-amino-2,6-dichlorophenol was condensed with 3,6, dichloro-4-isopropylpyridazine in presence of an inorganic base to produce 4-(6-chloro-5-isopropylpyridazin-3-yloxy)-3,5-dichlorobenzenamine, which is further oxidized in presence of NaOAc/AcOH to obtain 6-(4-amino-2,6-dichlorophenoxy)-4-isopropylpyridazin-3(2H)-one. However, this process results in the compound, 6-(4-amino-2,6-dichlorophenoxy)-4-isopropylpyridazin-3(2H)-one, with a yield of only 57% along with other byproducts, due to which, the resultant compound needed to be isolated using column chromatography.

In an alternate synthetic route, WO'913 discloses synthesis of phthalimide protected compound 65, which is prepared by condensing 4-amino-2,6-dichloro-phenol, with 3,6, dichloro-4-isopropylpyridazine using potassium tert-butoxide in N,N-dimethylacetamide at elevated temperature. The resulting intermediate was then treated with phthalic anhydride at elevated temperatures to form the phthalimide. The resulting phthalimide was heated in glacial acetic acid with sodium acetate to produce phthalimide protected compound 65 in 68% yields, as shown in scheme 2 below. However, this compound was further N-alkylated before subjecting to the hydrolysis to obtain 6-(4-Amino-2,6-dichloro-phenoxy)-4-isopropyl-2-methyl-2H-pyridazin-3-one, in a low yield of 74%. Moreover, the phthalic anhydride used for the amine protection is a toxic chemical; the use of the same in the pharmaceutical industry is not recommended.

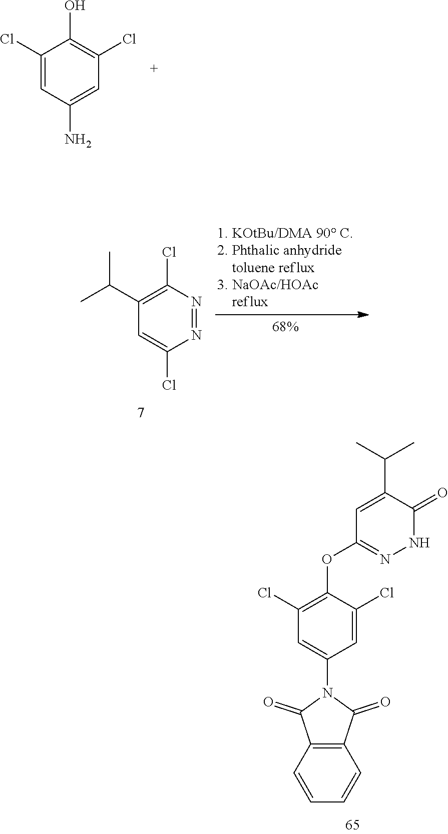 [SMILES: Nc1cc(Cl)c(O)c(Cl)c1 ; CC(C)c1cc(Cl)nnc1Cl ; CC(C)c1cc(Oc2c(Cl)cc(N3C(=O)c4ccccc4C3=O)cc2Cl)n[nH]c1=O]

While the process disclosed above as in scheme 1 results in the desired key intermediate in lower yields, which further necessitates the column/flash chromatography for the isolation of the 6-(4-amino-2,6-dichlorophenoxy)-4-isopropylpyridazin-3(2H)-one; the process disclosed under scheme 2 results in N-alkylated6-(4-amino-2,6-dichlorophenoxy)-4-isopropylpyridazin-3(2H)-one, viz., (6-(4-Amino-2,6-dichloro-phenoxy)-4-isopropyl-2-methyl-2H-pyridazin-3-one) in lower yields (as shown in reference example in the specification). Moreover, use of toxic chemicals such as phthalic anhydride for industrial production is not feasible and hence these reported processes are not viable for industrial scale up.

WO2014043706 discloses an alternate synthetic route for the preparation of 6-(4-amino-2,6-dichlorophenoxy)-4-isopropylpyridazin-3(2H)-one by the process shown in scheme 3 below.

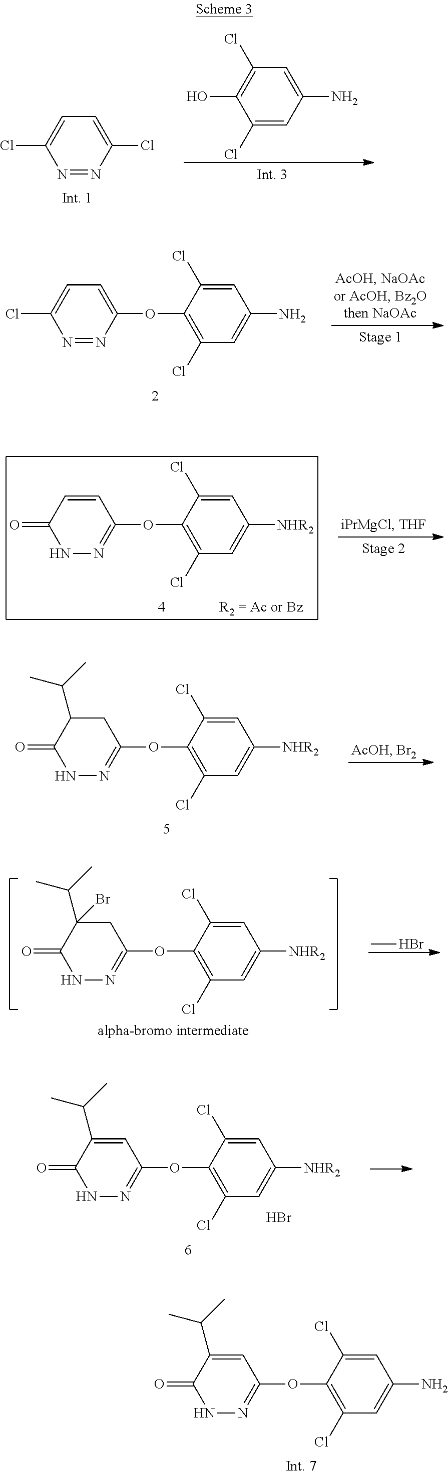

According to the above synthesis, the compound 2 is prepared by reacting 3,6-dichloropyridazine with 2,6-dichloro-4-aminophenol in the presence of a suitable base such as a metal or a metal alkoxide in a suitable organic solvent at a reaction temperature of 60° C. to 120° C. The compound 4 is prepared by protecting the amine group of the compound 2 with a benzoic anhydride or benzoyl chloride followed by treating the benzyl or benzoyl protected intermediate with sodium acetate in acetic acid to obtain compound 4. Compound 6 is prepared by reacting the compound 4 with isopropyl Grignard reagent followed by oxidation in presence of bromine and acetic acid at a reaction temperature of 60-90° C. Compound 7 is obtained by deprotecting Compound 6 (where R2 is Bz) with a base such as metal hydroxide or metal carbonate or deprotecting Compound 6 (where R2 is Ac) with an acid such as trifluoroacetic acid.

The above process results in huge variation in the yields and purity of the process intermediates, as provided under tables 5, 8, 9 and 10. The results provided in these tables clearly exemplify the reaction inconsistency and unpredictability. Moreover, the above process involves additional process steps and toxic chemical such as bromine which is not eco-friendly, to carry out the process on industrial scale.

Therefore, there remains a need in the art to provide an improved process for the synthesis of Res metirom by preparing the key intermediate, 6-(4-amino, 2,6-dichlorophenoxy)4-isopropylpyridazin-3(2H)-one in cost-effective manner.

### OBJECTIVES OF THE INVENTION

It is an objective of the present invention to provide a cost-effective process for the preparation of Re smetirom via a novel intermediate compounds of formula A.

It is another objective of the present invention to provide a cost-effective process for the preparation of a key intermediate, 6-(4-amino, 2,6-dichlorophenoxy)4-isopropylpyridazin-3(2H)-one, of Res metirom via a novel intermediate compounds of formula A.

It is a further objective of the invention to provide the novel intermediate compounds of formula A.

### SUMMARY OF THE INVENTION

In line with the above objectives, the present invention provides a process for synthesis of 6-(4-amino, 2,6-dichlorophenoxy)4-isopropylpyridazin-3(2H)-one, a key intermediate of Res metirom via a novel intermediate compounds of formula A, which process comprises;
a) Reacting 4-(6-chloro-5-isopropylpyridazin-3-yloxy)-3,5-dichlorobenzenamine with an acid anhydride of formula (RC(═O)OC(═O)R, or acid chloride of formula, RCOCl, (wherein R is methyl, ethyl or propyl) to obtain intermediate compounds of formula A;
b) Oxidizing the intermediate compounds of formula A in presence of NaOAc/AcOH to obtain intermediate compound of formula B; and
c) Hydrolysing the intermediate compound of formula B to obtain 6-(4-amino-2,6-dichlorophenoxy)-4-isopropylpyridazin-3(2H)-one.

In yet another aspect, the invention provides process for preparation of intermediate compounds of formula A, novel intermediates, which process comprises reaction of 4-(6-chloro-5-isopropylpyridazin-3-yloxy)-3,5-dichlorobenzenamine with acid anhydride of formula (RC(═O)OC(═O)R, or acid chloride of formula, RCOCl, (wherein R is methyl, ethyl or propyl) to obtain intermediate compounds of formula A (wherein, R is methyl, ethyl or propyl) respectively.

In yet another aspect, the starting compound, viz., 4-(6-chloro-5-isopropylpyridazin-3-yloxy)-3,5-dichlorobenzenamine can be prepared by the method reported in WO2007009913 by condensation of 4-amino-2,6-dichlorophenol with 3,6, dichloro-4-isopropylpyridazine in presence of a suitable base to produce 4-(6-chloro-5-isopropylpyridazin-3-yloxy)-3,5-dichlorobenzenamine.

In another aspect, the invention provides novel intermediate compounds of formula A,

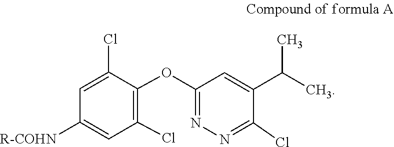

Wherein, R is selected from the group consisting of methyl, ethyl, propyl, etc.

In one of the specific embodiments, the intermediate compound of formula A1, wherein R is methyl, characterised by m/z 376.31.

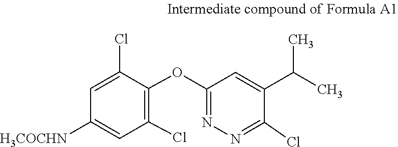

In another aspect, the intermediate compounds of formula A are isolated and characterised.

In another aspect, the novel intermediate compounds of formula A, are not isolated, however, further converted in-situ into intermediate compound of formula B, which upon hydrolysis results in 6-(4-amino-2,6-dichlorophenoxy)-4-isopropylpyridazin-3(2H)-one.

The 6-(4-amino-2,6-dichlorophenoxy)-4-isopropylpyridazin-3(2H)-one thus obtained is further reacted with N-cyanoacetyl urethane in presence of sodium nitrite and sodium acetate to obtain (2-Cyano-2-{[3,5-dichloro-4-(5-isopropyl-1-methyl-6-oxo-1,6-dihydro-pyridazin-3-yloxy)-phenyl]-hydrazono}-acetyl)-carbamic acid ethyl ester, which upon treatment with sodium acetate or potassium acetate to obtain 2-[3,5-Dichloro-4-(5-isopropyl-1-methyl-6-oxo-1,6-dihydro-pyridazin-3-yloxy)-phenyl]-3,5-dioxo-2,3,4,5-tetrahydro-[1,2,4]triazine-6-carbonitrile (Res metirom), following the conventional method.

Accordingly, in another aspect, the present invention provides an improved process for preparation of Res metirom via novel intermediate compounds of formula A, characterised in that;
a) Reacting the 4-(6-chloro-5-isopropylpyridazin-3-yloxy)-3,5-dichlorobenzenamine with an acid anhydride of formula (RC(═O)OC(═O)R or an acid chloride of formula RCOCl, wherein R is methyl, ethyl or propyl) to obtain intermediate compound of formula A;
b) Oxidizing the intermediate compounds of formula A in presence of NaOAc/AcOH to obtain intermediate compound of formula B;
c) Hydrolysing the intermediate compound of formula B to obtain 6-(4-amino-2,6-dichlorophenoxy)-4-isopropylpyridazin-3(2H)-one;
d) Reacting the 6-(4-amino-2,6-dichlorophenoxy)-4-isopropylpyridazin-3(2H)-one with N-cyanoacetyl urethane in presence of sodium nitrite and sodium acetate to obtain (2-Cyano-2-{[3,5-dichloro-4-(5-isopropyl-1-methyl-6-oxo-1,6-dihydro-pyridazin-3-yloxy)-phenyl]-hydrazono}-acetyl)-carbamic acid ethyl ester; and
e) Reacting the (2-Cyano-2-{[3,5-dichloro-4-(5-isopropyl-1-methyl-6-oxo-1,6-dihydro-pyridazin-3-yloxy)-phenyl]-hydrazono}-acetyl)-carbamic acid ethyl ester with sodium acetate or potassium acetate to obtain 2-[3,5-Dichloro-4-(5-isopropyl-1-methyl-6-oxo-1,6-dihydro-pyridazin-3-yloxy)-phenyl]-3,5-dioxo-2,3,4,5-tetrahydro-[1,2,4]triazine-6-carbonitrile.

### DETAILED DESCRIPTION OF THE INVENTION

The invention will now be described in detail in connection with certain preferred and optional embodiments, so that various aspects thereof may be more fully understood and appreciated.

The present invention provides a process for synthesis of 6-(4-amino, 2,6-dichlorophenoxy)4-isopropylpyridazin-3(2H)-one, a key intermediate of Res metirom via novel intermediate compounds of formula A, which process comprises;
a) Reacting 4-(6-chloro-5-isopropylpyridazin-3-yloxy)-3,5-dichlorobenzenamine with an acid anhydride of formula (RC(═O)OC(═O)R or an acid chloride of formula RCOCl, wherein R is methyl, ethyl or propyl) to obtain intermediate compounds of formula A;
b) Oxidizing the intermediate compounds of formula A in presence of NaOAc/AcOH to obtain a compound of formula B; and
c) Hydrolysing the compound of formula B to obtain 6-(4-amino-2,6-dichlorophenoxy)-4-isopropylpyridazin-3(2H)-one.

The acid anhydrides of general formula RC(═O)OC(═O)R, is used in the synthesis of intermediate compounds of formula A, wherein, R is selected from the group consisting of methyl, ethyl and propyl. Consequently, the acid anhydrides are selected from acetic anhydride, propionic anhydride and butyric anhydride.

Similarly, the reaction of 4-(6-chloro-5-isopropylpyridazin-3-yloxy)-3,5-dichlorobenzenamine with an acid chloride of formula RCOCl, is carried out conveniently to obtain compounds of Formula A, wherein R is methyl, ethyl or propyl. Consequently, the acid chlorides are selected from acetyl chloride, propanoyl chloride and butanoyl chloride.

In yet another embodiment, the invention provides process for preparation of a novel intermediate compound of formula A, which process comprises; reaction of 4-(6-chloro-5-isopropylpyridazin-3-yloxy)-3,5-dichlorobenzenamine with acetic acid/acetic anhydride or propanoic acid/propionic anhydride or butanoic acid/butyric anhydride to obtain novel intermediate compounds of formula A, respectively.

In yet another embodiment, the invention provides process for preparation of a novel intermediate compound of formula A, which process comprises; reaction of 4-(6-chloro-5-isopropylpyridazin-3-yloxy)-3,5-dichlorobenzenamine with an acid chloride selected from acetyl chloride, propanoyl chloride and butanoyl chloride, to obtain novel intermediate compounds of formula A, respectively.

In another embodiment, the invention provides novel intermediate compounds of formula A,

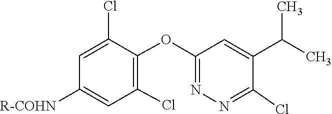

Wherein, R is selected from the group consisting of methyl, ethyl, propyl, etc.

### Compound of Formula A

In a more preferred embodiment, the invention provides compound of formula A1, wherein, R is methyl, characterized by m/z 376.31.

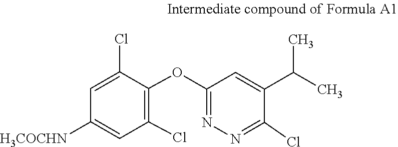

Accordingly, in one of the preferred embodiments, 4-(6-chloro-5-isopropylpyridazin-3-yloxy)-3,5-dichlorobenzenamine is reacted with acetic acid/acetic anhydride at a temperature range of 30 to 70° C. for 2 to 4 hrs. After the completion of the reaction by TLC, the intermediate compound of formula A1 (wherein, R is methyl) thus obtained was isolated by addition of water, filtration and further purification in Methanol.

Accordingly, in a further embodiment, the novel intermediate compound of formula A1 (wherein, R is methyl) thus obtained was characterised by subjecting to Mass (376.31 m/z in positive mode using Electro spray ionization (ESI) technique), HNMR, C13 NMR and FT IR.

In another preferred embodiment, 4-(6-chloro-5-isopropylpyridazin-3-yloxy)-3,5-dichlorobenzenamine is reacted with propanoic acid/propionic anhydride at a temperature range of 30 to 70° C. for 2 to 4 hrs. After the completion of the reaction by TLC, the intermediate compound of formula A2 (wherein, R is ethyl) thus obtained was isolated by addition of water, filtration and further purification in Methanol. However, the isolated compound was not characterised.

In another preferred embodiment, 4-(6-chloro-5-isopropylpyridazin-3-yloxy)-3,5-dichlorobenzenamine is reacted with butanoic acid/butyric anhydride at a temperature range of 30 to 70° C. for 2 to 4 hrs. After the completion of the reaction by TLC, the intermediate compound of formula A3 (wherein, R is ethyl) thus obtained was isolated by addition of water, filtration and further purification in Methanol. However, the isolated compound was not characterised.

In yet another preferred embodiment, 4-(6-chloro-5-isopropylpyridazin-3-yloxy)-3,5-dichlorobenzenamine is reacted with acetyl chloride at a temperature range of 30 to 70° C. for 2 to 4 hrs. After the completion of the reaction by TLC, the intermediate compound of formula A1 (wherein, R is methyl) thus obtained was isolated by addition of water, filtration and further purification in Methanol.

In yet another embodiment, the novel intermediate compound of formula A (wherein, R is methyl/ethyl/propyl), was not isolated, however, further converted in-situ by reacting with sodium acetate at a temperature range of 90 to 130° C. for 8 to 10 hrs, to obtain intermediate compound of formula B, as shown below.

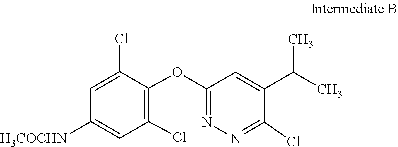

The intermediate compound B upon hydrolysis in presence of a mineral acid and an alcohol of C1 to C5 at room temperature results in 6-(4-amino-2,6-dichlorophenoxy)-4-isopropylpyridazin-3(2H)-one. The alcohols of C1 to C5 may be selected from the group consisting of methanol, ethanol, propanol, butanol, isobutanol, etc.

The mineral acid may be selected from the group consisting of sulphuric acid, hydrochloric acid, nitric acid and phosphoric acid.

The intermediate compound of formula B was first disclosed in WO2014043706.

The protection of 4-(6-chloro-5-isopropylpyridazin-3-yloxy)-3,5-dichlorobenzenamine with acid anhydride or an acid chloride is a neat reaction to obtain intermediate compound of formula A, as provided in the present invention, facilitates clean reactions subsequently with high yields and high purity viz., oxidation of intermediate A to obtain intermediate compound of formula B and hydrolysis of intermediate compound B to obtain 6-(4-amino-2,6-dichlorophenoxy)-4-isopropylpyridazin-3(2H)-one. The amino protection of 4-(6-chloro-5-isopropylpyridazin-3-yloxy)-3,5-dichlorobenzenamine with acetyl group substantially eliminates impurities formation during the production of intermediate compound B and upon hydrolysis of intermediate compound B, results in 6-(4-amino-2,6-dichlorophenoxy)-4-isopropylpyridazin-3(2H)-one with higher yields and purity, as demonstrated in the present invention. Thus the present invention eliminates the additional purification process steps unlike the processes reported in the prior arts.

The process of the present invention is shown in scheme 4 below.

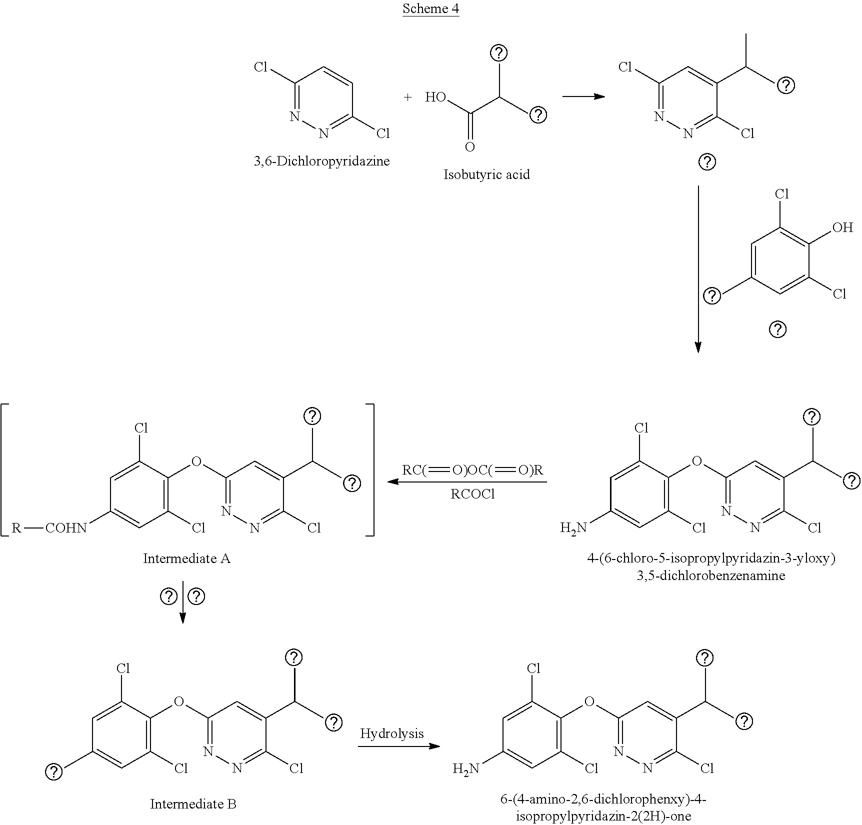

Wherein, R is selected from methyl, ethyl or propyl.

In yet another embodiment, the 6-(4-amino-2,6-dichlorophenoxy)-4-isopropylpyridazin-3(2H)-one thus obtained was further reacted with N-cyanoacetyl urethane in presence of sodium nitrite and sodium acetate to obtain (2-Cyano-2-{[3,5-dichloro-4-(5-isopropyl-1-methyl-6-oxo-1,6-dihydro-pyridazin-3-yloxy)-phenyl]-hydrazono}-acetyl)-carbamic acid ethyl ester, which upon treatment with sodium acetate or potassium acetate to obtain 2-[3,5-Dichloro-4-(5-isopropyl-1-methyl-6-oxo-1,6-dihydro-pyridazin-3-yloxy)-phenyl]-3,5-dioxo-2,3,4,5-tetrahydro-[1,2,4]triazine-6-carbonitrile, following the conventional method, as shown in scheme 5 below.

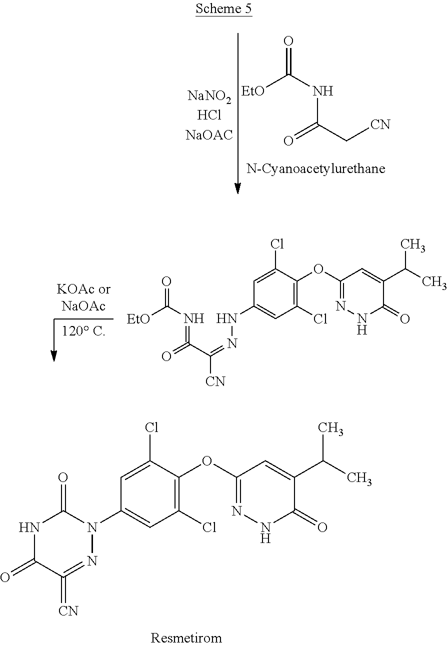

Accordingly, in yet another embodiment, the present invention provides an improved process for preparation of Res metirom via a novel intermediate compound of formula A, characterised in that;
a) reacting 4-(6-chloro-5-isopropylpyridazin-3-yloxy)-3,5-dichlorobenzenamine with an acid anhydride of formula (RC(═O)OC(═O)R or an acid chloride of formula RCOCl, wherein R is methyl, ethyl or propyl) to obtain intermediate compound of formula A;
b) Oxidizing the intermediate compound of formula A in presence of NaOAc/AcOH to obtain intermediate compound of B;
c) Hydrolysing the intermediate compound of B to obtain 6-(4-amino-2,6-dichlorophenoxy)-4-isopropylpyridazin-3 (2H)-one;
d) Reacting the 6-(4-amino-2,6-dichlorophenoxy)-4-isopropylpyridazin-3(2H)-one with N-cyanoacetyl urethane in presence of sodium nitrite and sodium acetate to obtain (2-Cyano-2-{[3,5-dichloro-4-(5-isopropyl-1-methyl-6-oxo-1,6-dihydro-pyridazin-3-yloxy)-phenyl]-hydrazono}-acetyl)-carbamic acid ethyl ester; and
e) Reacting the (2-Cyano-2-{[3,5-dichloro-4-(5-isopropyl-1-methyl-6-oxo-1,6-dihydro-pyridazin-3-yloxy)-phenyl]-hydrazono}-acetyl)-carbamic acid ethyl ester with sodium acetate or potassium acetate to obtain 2-[3,5-Dichloro-4-(5-isopropyl-1-methyl-6-oxo-1,6-dihydro-pyridazin-3-yloxy)-phenyl]-3,5-dioxo-2,3,4,5-tetrahydro-[1,2,4]triazine-6-carbonitrile.

The following example, which includes preferred embodiments, will serve to illustrate the practice of this invention, it being understood that the particulars shown are by way of example and for purpose of illustrative discussion of preferred embodiments of the invention.

### EXAMPLES

### Example 1

### Preparation of Intermediate Compound B from 4-(6-chloro-5-isopropylpyridazin-3-yloxy)-3,5-dichlorobenzenamine

Added 30 gm of 4-(6-chloro-5-isopropylpyridazin-3-yloxy)-3,5-dichlorobenzenamine into acetic acid (428 ml), added acetic anhydride (12.8 ml) and maintained for 2 hrs at 50 deg C. The progression of the reaction was monitored by TLC. After the completion of the reaction (TLC shows the absence of 4-(6-chloro-5-isopropylpyridazin-3-yloxy)-3,5-dichlorobenzenamine and formation of intermediate compound A), added Sodium Acetate (27.4 gram), heated the reaction mass to 120 Deg C, maintained at 120 deg C. for 8 to 10 hrs. The progression of the reaction was monitored by TLC. After the completion of the reaction (TLC shows the 1 to 3% of intermediate compound A and formation of intermediate compound B), added Water (440 ml), Cooled to RT, filtered the Solids and washed with Water (50 ml) to obtain the intermediate compound B.

Wet wt.=27.5 grams, Dry Wt=25 Grams. (Purity by HPLC>95%, % Yield=78%)

### Example 2

### Preparation of 6-(4-amino-2,6-dichlorophenoxy)-4-isopropylpyridazin-3(2H)-one from Intermediate B

Added intermediate B (24 gms) to methanol (550 ml) and added sulfuric Acid (24 grams) at RT. The reaction mass was stirred till the reaction is completed by TLC. Distilled out Methanol, added Water and filtered the Solids. The solid thus obtained was taken in water, adjusted pH between 7 to 8 using aqueous soda ash. The solids thus obtained were filtered and washed with water to obtain 6-(4-amino-2,6-dichlorophenoxy)-4-isopropylpyridazin-3(2H)-one.

Wet Wt=24 Grams, Dry wt=18 grams (Purity by HPLC>97, % Yield=86%)

### Example 3

### Preparation of Intermediate Compound A1, Wherein, R is Methyl

Added 30 gm of 4-(6-chloro-5-isopropylpyridazin-3-yloxy)-3,5-dichlorobenzenamine into acetic acid (428 ml), added acetic anhydride (12.8 ml) and maintained for 2 hrs at 50 deg C. The progression of the reaction was monitored by TLC. After the completion of the reaction (TLC shows the absence of 4-(6-chloro-5-isopropylpyridazin-3-yloxy)-3,5-dichlorobenzenamine and formation of intermediate compound A), Further the product was isolated by adding Water and Filtered. The Crude product was dissolved in Methanol (100 ml). Filtered over Charcoal and re-precipitated by Cooling and filtered.

Wet weight: 30 grams

Dry weight: 26.4 grams

Yield: 85%

Melting Point: 210 to 215 deg C.

Mass: 376.31 m/z

Purity: >99%

### Example 4

### Preparation of Intermediate Compound A1, Wherein, R is Methyl

Added 15 gm of 4-(6-chloro-5-isopropylpyridazin-3-yloxy)-3,5-dichlorobenzenamine into acetic acid (214 ml), added acetic anhydride (6.4 ml) and maintained for 2 hrs at 50 deg C. The progression of the reaction was monitored by TLC. After the completion of the reaction (TLC shows the absence of 4-(6-chloro-5-isopropylpyridazin-3-yloxy)-3,5-dichlorobenzenamine and formation of intermediate compound A), Further the product was isolated by adding Water and Filtered. The Crude product was dissolved in Methanol (50 ml). Filtered over Charcoal and re-precipitated by Cooling and filtered.

Wet weight: 16 grams

Dry weight: 14.4 grams

Yield: 88%

Purity: >99%

### Example 5

### Preparation of Intermediate Compound A2, Wherein, R is Ethyl

Added 30 gm of 4-(6-chloro-5-isopropylpyridazin-3-yloxy)-3,5-dichlorobenzenamine into propanoic acid (428 ml), added propionic anhydride (15.8 ml) and maintained for 2 hrs at 50 deg C. The progression of the reaction was monitored by TLC. After the completion of the reaction (TLC shows the absence of 4-(6-chloro-5-isopropylpyridazin-3-yloxy)-3,5-dichlorobenzenamine and formation of intermediate compound A), Further the product was isolated by adding Water and Filtered. The Crude product was dissolved in Methanol (100 ml). Filtered over Charcoal and re-precipitated by Cooling and filtered.

Wet weight: 32 grams

Dry weight: 27.4 grams

Yield: 85%

Purity: >99%

### Example 6

### Preparation of Intermediate Compound A3, Wherein, R is Propyl

Added 30 gm of 4-(6-chloro-5-isopropylpyridazin-3-yloxy)-3,5-dichlorobenzenamine into butanoic acid (428 ml), added butyric anhydride (17.8 ml) and maintained for 2 hrs at 50 deg C. The progression of the reaction was monitored by TLC. After the completion of the reaction (TLC shows the absence of 4-(6-chloro-5-isopropylpyridazin-3-yloxy)-3,5-dichlorobenzenamine and formation of intermediate compound A), Further the product was isolated by adding Water and Filtered. The Crude product was dissolved in Methanol (100 ml). Filtered over Charcoal and re-precipitated by Cooling and filtered.

Wet weight: 37 grams

Dry weight: 30 grams

Yield: 85%

Purity: >99%

### Example 7

### Preparation of Intermediate Compound A1, Wherein, R is Methyl

Added 15 gm of 4-(6-chloro-5-isopropylpyridazin-3-yloxy)-3,5-dichlorobenzenamine added acetyl chloride (6.4 ml) and maintained for 2 hrs at 50 deg C. The progression of the reaction was monitored by TLC. After the completion of the reaction (TLC shows the absence of 4-(6-chloro-5-isopropylpyridazin-3-yloxy)-3,5-dichlorobenzenamine and formation of intermediate compound A), the product was isolated by adding Water and Filtered. The Crude product was dissolved in Methanol (50 ml). Filtered over Charcoal and re-precipitated by Cooling and filtered.

Wet weight: 16 grams

Dry weight: 14.4 grams

Yield: 88%

Purity: >99%

### Reference Example 1 (WO2007009913)

### Step 1: Preparation of 2-[3,5-Dichloro-4-(5-isopropyl-6-oxo-1,6-dihydro-pyridazin-3-yloxy)-phenyl]-isoindole-1,3-dione (65)

A mixture of 4-amino-2,6-dichloro-phenol (50 g, 280.8 mmol) and potassium tert-butoxide (33.16 g, 280.8 mmol) in N,N-dimethylacetamide (200 mL) was heated to 90° C. The resulting solution was then treated with 3,6-dichloro-4-isopropylpyridazine (55.31 g, 280.8 mmol). The reaction was heated at 90° C. for 17 h. At this time, the reaction was diluted with methyl tert-butyl ether (700 mL) and a saturated aqueous sodium chloride solution (800 mL). The organic layer was separated, washed with water (2×400 mL) and concentrated to a volume of ˜200 mL. This solution was diluted with toluene (800 mL) and then distilled to remove −300 mL of solvent. The remaining solution was cooled to 80° C. and was treated with phthalic anhydride (42.01 g, 280.8 mmol). This mixture was heated to reflux for 4 h while water was distilled azeotropically. At this time, the reaction was concentrated under vacuum to −200 mL, diluted with glacial acetic acid (800 mL) and then concentrated to remove −300 mL of solvent. The reaction was treated with sodium acetate (46.06 g, 561.6 mmol) and heated to reflux for 6 h. At this time, the reaction was cooled to room temperature and diluted with water (500 mL). This mixture was warmed to 60° C., stirred for 30 min and then cooled to room temperature. The resulting solid was collected by filtration, washed with a 1:1 mixture of glacial acetic acid:water (300 mL) followed by water (150 mL), dried under house vacuum and then dried at 55° C. in a vacuum oven overnight to afford 2-[3,5-dichloro-4-(5-isopropyl-6-oxo-1,6-dihydro-pyridazin-3-yloxy)-phenyl]-isoindole-1,3-dione (84.8 g, 68%) as an off-white solid.

### Step 3: Preparation of 6-(4-Amino-2,6-dichloro-phenoxy)-4-isopropyl-2-methyl-2H-pyridazin-3-one (67)

A mixture of 2-[3,5-dichloro-4-(5-isopropyl-1-methyl-6-oxo-1,6-dihydro-pyridazin-3-yloxy)-phenyl]-isoindole-1,3-dione (66) (64 g, 139.6 mmol) in methanol (500 mL) was treated with butylamine (34.67 mL, 349 mmol). The mixture was heated to reflux for 1.5 h. At this time, the reaction was cooled to room temperature and treated dropwise with water (384 mL). The resulting solids were collected by filtration, washed with a 1:1 solution of methanol/water (180 mL) followed by water (250 mL) and dried under vacuum to afford 6-(4-amino-2,6-dichloro-phenoxy)-4-isopropyl-2-methyl-2H-pyridazin-3-one (67) (34.12 g, 74.4%) as an off-white solid.

1. A process for synthesis of 6-(4-amino, 2,6-dichlorophenoxy)4-isopropylpyridazin-3(2H)-one via an intermediate compound of formula A, the process comprising:
a) reacting 4-(6-chloro-5-isopropylpyridazin-3-yloxy)-3,5-dichlorobenzenamine with an acid anhydride of formula RC(═O)OC(═O)R, wherein R is a methyl, ethyl, or propyl, or an acid chloride of formula RCOCl, wherein R is methyl, ethyl or propyl, to obtain the intermediate compound of formula A;
b) oxidizing the intermediate compound of formula A in the presence of NaOAc/AcOH to obtain a compound of formula B; and
c) hydrolysing the compound of formula B in the presence of an alcohol and a mineral acid to obtain 6-(4-amino-2,6-dichlorophenoxy)-4-isopropylpyridazin-3(2H)-one.

2. The process as claimed in claim 1, wherein the acid anhydride comprises acetic anhydride, propionic anhydride or butyric anhydride.

3. The process as claimed in claim 1, wherein the reacting of 4-(6-chloro-5-isopropylpyridazin-3-yloxy)-3,5-dichlorobenzenamine with the acid anhydride is carried out at a temperature ranging from 30 to 70° C.

4. The process as claimed in claim 1, wherein the acid chloride comprises acetyl chloride, propanoyl chloride or butanoyl chloride.

5. The process as claimed in claim 1, wherein the oxidizing of the intermediate compound of formula A is carried out at a temperature ranging from 90 to 130° C.

6. The process as claimed in claim 1, wherein the mineral acid is comprises sulphuric acid, hydrochloric acid, nitric acid or phosphoric acid, and wherein the alcohol is selected from a C1 to C5 alcohols.

7. An intermediate compound of formula A having the following structure: 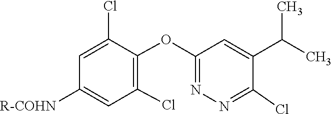
wherein R comprises a methyl, an ethyl or propyl group.

8. A process for preparation of the intermediate compound of formula A as claimed in claim 7, wherein the process comprises: reacting 4-(6-chloro-5-isopropylpyridazin-3-yloxy)-3,5-dichlorobenzenamine with an acid anhydride of formula RC(═O)OC(═O)R or an acid chloride of formula RCOCl, wherein R is methyl, ethyl or propyl, at a temperature range of 30 to 70° C. to obtain the intermediate compound of formula A.

9. The process as claimed in claim 8, wherein the acid anhydride comprises acetic anhydride, propionic anhydride or butyric anhydride.

10. The process as claimed in claim 8, wherein the acid chloride comprises acetyl chloride, propanoyl chloride or butanoyl chloride.

11. An intermediate compound of formula A1 having the following structure: 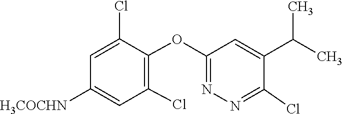
wherein the intermediate compound of formula A1 is characterized by a m/z of 376.31.

12. A process for preparation of the intermediate compound of formula A1 as claimed in claim 11, wherein the process comprises: reacting 4-(6-chloro-5-isopropylpyridazin-3-yloxy)-3,5-dichlorobenzenamine with acetic acid, acetic anhydride or acetyl chloride at a temperature ranging from 30 to 70° C. to obtain the intermediate compound of formula A1.

13. A process for preparation of Res-metirom via an intermediate compound of formula A, the process comprising:
a) reacting 4-(6-chloro-5-isopropylpyridazin-3-yloxy)-3,5-dichlorobenzenamine with an acid anhydride of formula RC(═O)OC(═O)R or an acid chloride of formula RCOCl, wherein R is methyl, ethyl or propyl, to obtain the intermediate compound of formula A;
b) oxidizing the intermediate compound of formula A in the presence of NaOAc/AcOH to obtain an intermediate compound of formula B;
c) hydrolysing the intermediate compound of formula B in the presence of an alcohol and a mineral acid to obtain 6-(4-amino-2,6-dichlorophenoxy)-4-isopropylpyridazin-3(2H)-one;
d) reacting the 6-(4-amino-2,6-dichlorophenoxy)-4-isopropylpyridazin-3(2H)-one with N-cyanoacetyl urethane in the presence of sodium nitrite and sodium acetate to obtain (2-cyano-2-{[3,5-dichloro-4-(5-isopropyl-1-methyl-6-oxo-1,6-dihydro-pyridazin-3-yloxy)-phenyl]-hydrazono}-acetyl)-carbamic acid ethyl ester; and
e) reacting the (2-cyano-2-{[3,5-dichloro-4-(5-isopropyl-1-methyl-6-oxo-1,6-dihydro-pyridazin-3-yloxy)-phenyl]-hydrazono}-acetyl)-carbamic acid ethyl ester with sodium acetate or potassium acetate to obtain 2-[3,5-dichloro-4-(5-isopropyl-1-methyl-6-oxo-1,6-dihydro-pyridazin-3-yloxy)-phenyl]-3,5-dioxo-2,3,4,5-tetrahydro-[1,2,4]triazine-6-carbonitrile.

14. The process as claimed in claim 13, wherein the acid anhydride comprises acetic anhydride, propionic anhydride or butyric anhydride.

15. The process as claimed in claim 13, wherein the reacting of 4-(6-chloro-5-isopropylpyridazin-3-yloxy)-3,5-dichlorobenzenamine with the acid anhydride is carried out at a temperature ranging from 30 to 70° C.

16. The process as claimed in claim 13, wherein the acid chloride comprises acetyl chloride, propanoyl chloride or butanoyl chloride.

17. The process as claimed in claim 13, wherein the oxidizing of the intermediate compounds of formula A is carried out at a temperature ranging from 90 to 130° C.

18. The process as claimed in claim 13, wherein the mineral acid comprises sulphuric acid, hydrochloric acid, nitric acid or phosphoric acid, and wherein the alcohol is selected from a C1 to C5 alcohols.
